(19) United States
(12) Patent Application Publication
(10) Pub. No.: US 2026/0221259 A1
(43) Pub. Date: Jul. 30, 2026

(54) SYSTEM, SERVER APPARATUS, CONTROL METHOD OF SERVER APPARATUS, AND NON-TRANSITORY COMPUTER-READABLE STORAGE MEDIUM

(71) Applicant: NEC Corporation, Tokyo (JP)

(72) Inventors: Atsuya MURASE, Tokyo (JP); Masahiro KIYOSAWA, Tokyo (JP); Mai YOSHIKAWA, Tokyo (JP); Yoshihisa YOSHIOKA, Tokyo (JP); Takahiro FURUKAWA, Tokyo (JP)

(73) Assignee: NEC Corporation, Tokyo (JP)

(21) Appl. No.: 19/444,319

(22) Filed: Jan. 9, 2026

(30) Foreign Application Priority Data
Jan. 30, 2025 (JP) 2025-014418

(51) Int. Cl. G16H 20/30; G06Q 40/08; G16H 20/60; H04L 9/32

(52) U.S. Cl. CPC G16H 20/30; G06Q 40/0821; G16H 20/60; H04L 9/3231; H04L 9/3268

(57) ABSTRACT

A system includes an issuance means, an acquisition means, a verification means, and an output means. The issuance means issues, to a user, a certificate that certifies predetermined matters in a VCs (Verifiable Credentials) format. The acquisition means acquires the certificate issued to the user in a case where the user requests provision of advice from a service provider. The verification means verifies the acquired certificate. The output means generates, in a case where the verification of the certificate succeeds, advice based on predetermined matters that the certificate certifies, and outputs the generated advice.

### INCORPORATION BY REFERENCE

This application is based upon and claims the benefit of priority from Japanese Patent Application No. 2025-014418, filed on January 30, 2025, the disclosure of which is incorporated herein in its entirety by reference.

### TECHNICAL FIELD

The present disclosure relates to a system, a server apparatus, a control method of a server apparatus, and a non-transitory computer-readable storage medium.

### BACKGROUND ART

A system that provides advice such as health management to a user exists.

For example, Patent Literature 1 (JP2008-059320A) describes a health management system capable of reducing the workload for creating advice on the advisor side regarding health management, and enabling a user to promptly receive advice at a desired time.

In the system of Patent Literature 1, a user terminal and a health management server are connected via a network. The health management server includes a health information receiving means, an advice information generating means, an advice information storing means, and an advice information transmitting means.

The health information receiving means receives health information from the user terminal. The advice information generating means generates advice information based on the health information. The advice information storing means stores the generated advice information. The advice information transmitting means transmits the stored advice information to the user terminal. The user terminal includes a health information transmitting means that transmits health information to the health management server, an advice information receiving means that receives advice information from the health management server, and an advice information display means that displays the received advice information.

In order for a service provider to provide appropriate advice to a user, the information serving as the basis thereof must be reliable. For example, in a case where the health information described in Patent Literature 1 is not correct, correct advice is not provided to the user.

### SUMMARY

It is a main object of the present disclosure to provide a system, a server apparatus, a control method of a server apparatus, and a non-transitory computer-readable storage medium that contribute to providing more appropriate advice to a user.

According to a first aspect of the present disclosure, there is provided a system including: an issuance means that issues, to a user, a certificate that certifies predetermined matters in a VCs (Verifiable Credentials) format; an acquisition means that acquires the certificate issued to the user in a case where the user requests provision of advice from a service provider; a verification means that verifies the acquired certificate; and an output means that generates, in a case where verification of the certificate succeeds, the advice based on the predetermined matters that the certificate certifies, and outputs the generated advice.

According to a second aspect of the present disclosure, there is provided a server apparatus including: an acquisition means that acquires a certificate, the certificate certifying predetermined matters and being issued in a VCs (Verifiable Credentials) format, in a case where a user requests provision of advice from a service provider; a verification means that verifies the acquired certificate; and an output means that generates, in a case where verification of the certificate succeeds, the advice based on the predetermined matters that the certificate certifies, and outputs the generated advice.

According to a third aspect of the present disclosure, there is provided a control method of a server apparatus, the control method including: acquiring a certificate, the certificate certifying predetermined matters and being issued in a VCs (Verifiable Credentials) format, in a case where a user requests provision of advice from a service provider; verifying the acquired certificate; generating, in a case where verification of the certificate succeeds, the advice based on the predetermined matters that the certificate certifies; and outputting the generated advice.

According to a fourth aspect of the present disclosure, there is provided a non-transitory computer-readable storage medium storing a program causing a computer mounted on a server apparatus to perform processing for: acquiring a certificate, the certificate certifying predetermined matters and being issued in a VCs (Verifiable Credentials) format, in a case where a user requests provision of advice from a service provider; verifying the acquired certificate; generating, in a case where verification of the certificate succeeds, the advice based on the predetermined matters that the certificate certifies; and outputting the generated advice.

### BRIEF DESCRIPTION OF THE DRAWINGS

Exemplary features and advantages of the present disclosure will become apparent from the following detailed description when taken with the accompanying drawings in which:

FIG. 1 is a diagram for explaining an outline of an example embodiment;

FIG. 2 is a flowchart illustrating an operation of an example embodiment;

FIG. 3 is a diagram illustrating an example of an outline configuration of the information processing system according to an example embodiment of the present disclosure;

FIG. 4 is a diagram illustrating an example of a display of a terminal according to an example embodiment of the present disclosure;

FIG. 5 is a diagram for explaining an operation of the information processing system according to the example embodiment of the present disclosure;

FIG. 6 is a diagram for explaining an operation of the information processing system according to the example embodiment of the present disclosure;

FIG. 7 is a diagram illustrating an example of a processing configuration of the terminal according to the example embodiment of the present disclosure;

FIG. 8 is a flowchart illustrating an example of an operation of an identity verification unit according to the example embodiment of the present disclosure;

FIG. 9 is a flowchart illustrating an example of an operation of an acquisition control unit according to the example embodiment of the present disclosure;

FIG. 10 is a diagram illustrating an example of a processing configuration of a hospital server according to the example embodiment of the present disclosure;

FIG. 11 is a diagram illustrating an example of a processing configuration of an insurance server according to the example embodiment of the present disclosure;

FIG. 12 is a flowchart illustrating an example of an operation of an advice provision control unit according to the example embodiment of the present disclosure;

FIG. 13 is a diagram illustrating an example of a display of the terminal according to the example embodiment of the present disclosure;

FIG. 14 is a sequence diagram illustrating an example of an operation of the information processing system according to the example embodiment of the present disclosure;

FIG. 15 is a sequence diagram illustrating an example of an operation of the information processing system according to the example embodiment of the present disclosure; and

FIG. 16 is a diagram illustrating an example of a hardware configuration of the terminal according to the present disclosure.

### EXAMPLE EMBODIMENT

First, an outline of an example embodiment will be described. It should be noted that, in the following outline, various components are denoted by reference characters for the sake of convenience. That is, the following reference characters are used as examples to facilitate the understanding of the present disclosure. Thus, the description of the outline is not intended to impose any limitations. In addition, unless otherwise specified, an individual block illustrated in the drawings represents a configuration of a functional unit, not a hardware unit. An individual connection line between blocks in the drawings signifies both one-way and two-way directions. An arrow schematically illustrates a principal signal (data) flow and does not exclude bidirectionality. It should be noted that, in the present description and drawings, elements that can be described in a like way will be denoted by a like reference character, and redundant description thereof will be omitted as needed.

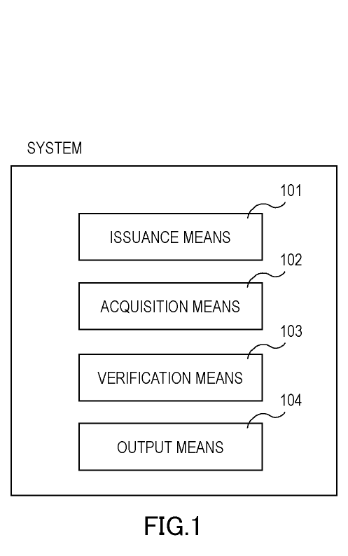

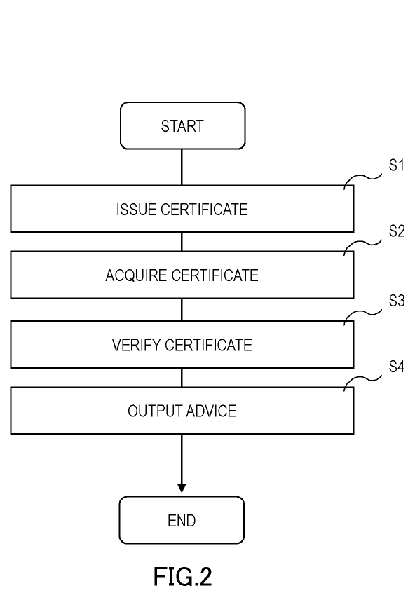

A system according to an example embodiment includes an issuance means 101, an acquisition means 102, a verification means 103, and an output means 104 (see FIG. 1). The issuance means 101 issues, to a user, a certificate that certifies predetermined matters in a VCs (Verifiable Credentials) format (issues the certificate; Step S1 of FIG. 2). The acquisition means 102 acquires the certificate issued to the user in a case where the user requests provision of advice from a service provider (Step S2). The verification means 103 verifies the acquired certificate (Step S3). The output means 104 generates, in a case where the verification of the certificate succeeds, advice based on predetermined matters that the certificate certifies, and outputs the generated advice (Step S4).

In the above system, the service provider provides advice to the user based on matters guaranteed by the certificate issued to the user (matters that the certificate certifies). As a result, more appropriate advice is provided to the user.

Hereinafter, specific example embodiments will be described in more detail with reference to drawings.

### First Example Embodiment

A first example embodiment will be described in more detail with reference to drawings.

In the information processing system according to the first example embodiment, a user can receive advice related to products and the like from a service provider. At that time, the service provider acquires, as an electronic certificate, information serving as a premise for the advice. The service provider provides, to the user, advice based on the acquired certificate in a case where the acquired certificate is valid.

In the first example embodiment, the service provider is a life insurance company, and a case will be described in which the life insurance company provides advice according to medical checkup results of the user.

### Configuration of the System

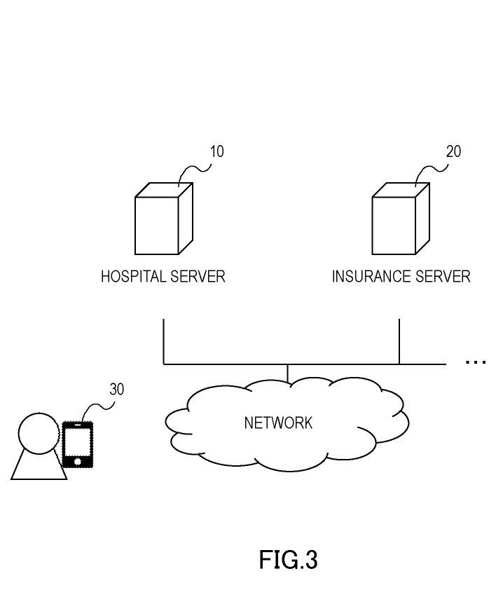

As illustrated in FIG. 3, the information processing system includes a hospital server 10 and an insurance server 20.

The hospital server 10 is a server that performs control related to hospital operations. The insurance server 20 is a server that performs control related to operations of a life insurance company. The hospital server 10 may be installed in a hospital or may be installed on a network (in the cloud). Similarly, the insurance server 20 may be installed in a building of a life insurance company or may be installed on a network (in the cloud).

A user possesses a terminal 30. For example, the user operates the terminal 30 to access the insurance server 20. The user inputs various information to the insurance server 20 via the terminal 30 and acquires various information from the insurance server 20.

Each apparatus shown in FIG. 3 is connected to a network. Specifically, the hospital server 10, the insurance server 20, and the terminal 30 are connected to the network by wired or wireless communication means.

The configuration of the information processing system shown in FIG. 3 is an example and is not intended to limit the configuration of the authentication system. For example, a hospital may include a plurality of hospital servers 10. Load balancing or redundancy may be realized by the plurality of hospital servers 10.

### Outline of operation

Subsequently, an outline operation of the information processing system according to a first example embodiment will be described.

### Preparation of Digital Wallet

The user's terminal 30 includes a digital wallet function. A digital wallet is an electronic information storage service whose information security properties such as data integrity, reliability, and availability are ensured.

A user installs an application (a digital wallet application) for implementing a digital wallet in the terminal 30 possessed by the user. By opening a digital wallet in the terminal 30, the user can store digital contents such as a digital employee ID, electronic money, identity documents such as a passport or driver's license, and various tickets such as airline tickets, in the terminal 30.

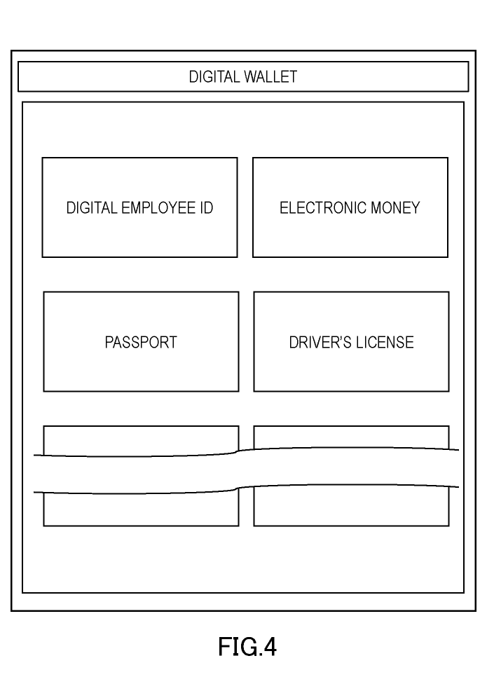

For example, the user's terminal 30 stores digital contents as illustrated in FIG. 4. Among the digital contents stored in the terminal 30 are public identity documents such as a passport and a driver's license, and digital employee IDs issued by a company.

### Implementation of Medical Checkup

A user undergoes a medical checkup at a hospital. Hospital staff and the like register information of the user who underwent the medical checkup in the hospital server 10.

For example, hospital staff register the user's name, gender, date of birth, address, patient ID number, telephone number, email address, and the like in the hospital server 10. In addition, hospital staff store the medical checkup results in association with the user's name and the like. The hospital server 10 generates an account of the user who underwent the medical checkup and stores, in the generated account (database), the name, biometric information, medical checkup results, and the like in association with one another.

### Acquisition of Medical Checkup Certificate

A user acquires digital contents to be stored in a digital wallet. For example, the user acquires a medical checkup certificate in which

the medical checkup results are guaranteed by the hospital. Specifically, the terminal 30 (digital wallet application) makes a request for issuance of a medical checkup certificate to the hospital at which the user underwent the medical checkup.

The hospital (the hospital server 10) that has received the request for issuance of the medical checkup certificate issues a certificate certifying the medical checkup results. Specifically, the hospital server 10 issues VCs (Verifiable Credentials), whose contents can be verified online, as the medical checkup certificate.

In the following description, VCs are referred to as "credential certificates," and a medical checkup certificate issued as VCs is referred to as "medical checkup certificate VCs."

Prior to a request for issuance of a credential certificate (medical checkup certificate VCs), the terminal 30 performs identity verification using an identity document issued by a public institution such as a governmental organization. For example, the terminal 30 performs identity verification of the user at the time of opening the digital wallet or at a timing at which the user desires issuance of the medical checkup certificate VCs.

For example, the terminal 30 performs identity verification using an identity document such as a My Number Card or a passport, in which biometric information of the holder is recorded. The terminal 30 uses a My Number Card or a passport as a root of trust.

It should be noted that examples of the biometric information include data (feature values) calculated from physical features unique to an individual, such as a face, a fingerprint, a voiceprint, a vein, a retina, or an iris pattern of an eye. Alternatively, the biometric information may be image data such as a face image or a fingerprint image. The biometric information may be anything that includes physical characteristics of a user as information. In the present disclosure, a case where biometric information relating to a human "face" (a face image or a feature value generated from the face image) is used will be described.

The terminal 30 acquires information related to the holder (the certificate holder) of the identity document from the user's identity document. For example, the terminal 30 acquires information related to the holder of the My Number Card from an IC (Integrated Circuit) of the My Number Card.

Specifically, the terminal 30 acquires biometric information (a face image) of the holder of the My Number Card. The terminal 30 stores, internally, the biometric information read from the My Number Card.

In addition, the terminal 30 acquires biometric information of the user. For example, the terminal 30 acquires and stores a face image by photographing the user.

The terminal 30 performs matching processing (authentication processing) using the biometric information acquired from the identity document and the biometric information obtained by photographing the user. The terminal 30 is able to make a request for issuance of the medical checkup certificate VCs in a case where the authentication processing (one-to-one authentication) succeeds.

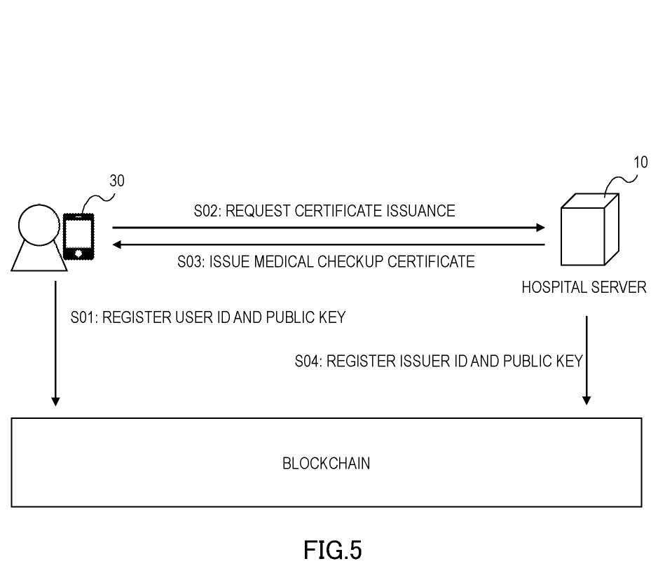

In addition, prior to a request for issuance of the medical checkup certificate VCs, the user's terminal 30 generates a pair of a public key and a private key. In addition, the terminal 30 generates a decentralized identifier (DID; Decentralized Identifier). The terminal 30 registers the generated DID (user ID; holder ID) and the public key in a blockchain (Step S01 of FIG. 5).

Further, the user's terminal 30, while presenting the user ID, makes a request for issuance of the medical checkup certificate VCs to the issuer of the medical checkup certificate VCs (the hospital, the hospital server 10). Specifically, the terminal 30 transmits a "certificate issuance request," which includes information for identifying the user (for example, a name, a combination of the name and date of birth, a patient ID number) and the user ID and the like, to the hospital server 10 (Step S02).

It should be noted that the hospital server 10 generates and stores an issuer DID (issuer ID), a private key, and a public key in advance.

In a case where the certificate issuance request is received, the hospital server 10 determines whether the user requesting issuance of the medical checkup certificate VCs is qualified to receive issuance of the medical checkup certificate VCs. Specifically, the hospital server 10 determines whether the user who desires issuance of the medical checkup certificate VCs is a user who underwent the medical checkup.

In a case where the user is qualified to receive issuance of the medical checkup certificate VCs, the hospital server 10 generates the medical checkup certificate VCs including an issuer ID and a user ID.

Specifically, the hospital server 10 generates the medical checkup certificate VCs including metadata such as a type of the credential certificate (medical checkup certificate), an issuing organization name, an issuance date and time, and the like, credential information itself, and information such as the issuer's public key information and an electronic signature. It should be noted that specific information certified by the issuer (for example, a name, medical checkup results, and the like) is written in the credential information itself.

The hospital server 10 transmits the generated medical checkup certificate VCs to the terminal 30 of the user (the user who becomes the holder of the medical checkup certificate VCs; the requester of issuance of the medical checkup certificate VCs) (issuance of the medical checkup certificate; Step S03).

Further, the hospital server 10 registers the issuer ID and the generated public key in a blockchain (Step S04).

The terminal 30 stores the received medical checkup certificate VCs in the digital wallet.

### Provision of Advice

A user can receive various types of advice from a life insurance company. Specifically, the user can receive advice related to insurance products (for example, advice related to proposals of insurance products or discounts of insurance premiums) and advice related to health such as lifestyle improvement.

At that time, the user presents a certificate required by the life insurance company. Specifically, the user provides the medical checkup certificate VCs to the life insurance company.

The user accesses the insurance server 20 by operating the terminal 30. The user makes an application for provision of advice on a website and the like provided by the insurance server 20. For example, the user selects a desired type of advice from menus such as "health advice," "product proposal," and the like.

In a case where a user requests provision of advice, the insurance server 20 requests the terminal 30 to provide the medical checkup certificate VCs.

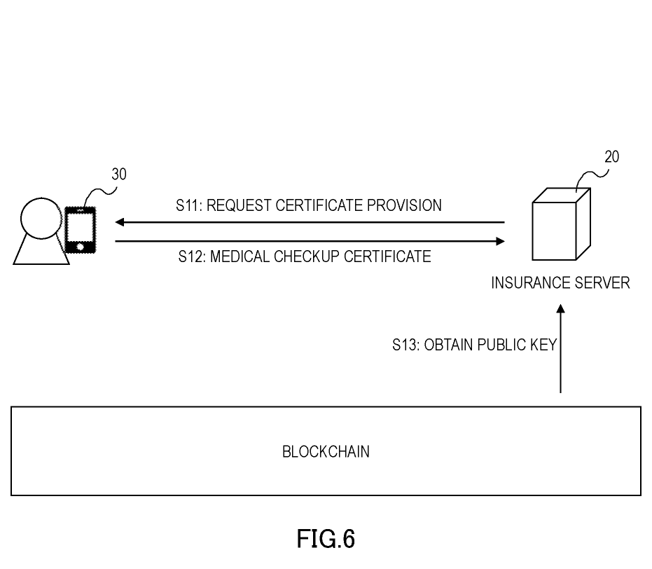

Specifically, the insurance server 20 transmits a "certificate provision request" to the terminal 30 (Step S11 of FIG. 6). In the certificate provision request, the medical checkup certificate VCs are set as information that the life insurance company requests to be provided.

In response to receipt of the certificate provision request, the terminal 30 transmits the medical checkup certificate VCs stored in the digital wallet to the insurance server 20. Specifically, the terminal 30 selects the designated certificate (the medical checkup certificate VCs) from among a plurality of certificates stored in the digital wallet.

Thereafter, the terminal 30 signs the selected medical checkup certificate VCs using a private key corresponding to the user's DID (user ID). The terminal 30 transmits the medical checkup certificate VCs with the signature to the insurance server 20 (Step S12).

The insurance server 20 verifies validity of the received medical checkup certificate VCs. At that time, the insurance server 20 acquires a public key from a blockchain (Step S13). It should be noted that details regarding verification of validity of the medical checkup certificate VCs will be described later.

In a case where the acquired medical checkup certificate VCs are valid, the insurance server 20 provides advice to the user. For example, the insurance server 20 displays the generated advice on the terminal 30.

Next, details of the individual apparatuses included in the information processing system according to the first example embodiment will be described.

### Terminal

Examples of the terminal 30 include a portable terminal device such as a smartphone, a portable phone, a game console, or a tablet and a computer (a personal computer or a laptop computer). The terminal 30 can be any equipment or device as long as the terminal 30 can accept an operation by a user and can communicate with the insurance server 20 and the like.

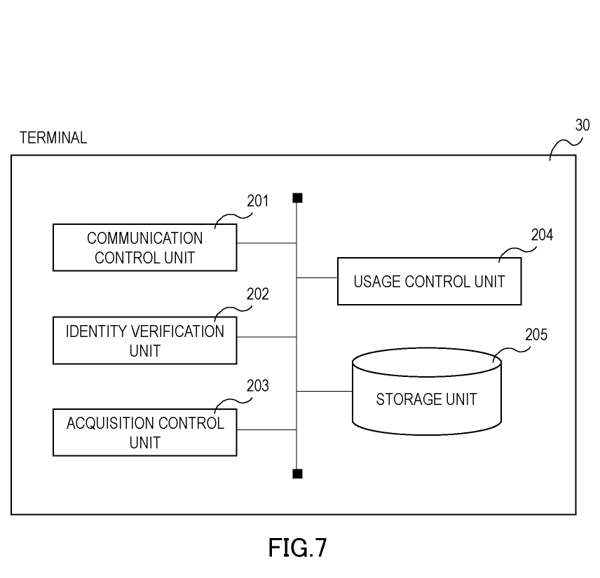

FIG. 7 is a diagram illustrating an example of a processing configuration (processing modules) of the terminal 30 according to an example embodiment of the present disclosure. Referring to FIG. 7, the terminal 30 includes a communication control unit 201, an identity verification unit 202, an acquisition control unit 203, a usage control unit 204, and a storage unit 205.

The communication control unit 201 is means for controlling communication with other apparatuses. For example, the communication control unit 201 receives data (packets) from the hospital server 10. In addition, the communication control unit 201 transmits data to the hospital server 10. The communication control unit 201 gives data received from other apparatuses to other processing modules. The communication control unit 201 transmits data acquired from other processing modules to other apparatuses. In this way, other processing modules transmit and receive data to and from other apparatuses via the communication control unit 201. The communication control unit 201 includes a function as a receiving unit that receives data from other apparatuses and a function as a transmitting unit that transmits data to other apparatuses.

The identity verification unit 202, the acquisition control unit 203, and the usage control unit 204 implement a digital wallet application. A detailed description regarding installation of the digital wallet application will be omitted. This is because installation of an application is obvious to those skilled in the art.

The identity verification unit 202 is a means for performing identity verification of a user who uses the digital wallet.

The identity verification unit 202 performs identity verification using biometric information obtained from an identity document and biometric information of a user who uses the digital wallet. More specifically, the identity verification unit 202 confirms that the user of the digital wallet and the holder (the certificate holder) of the identity document issued by a public institution are identical.

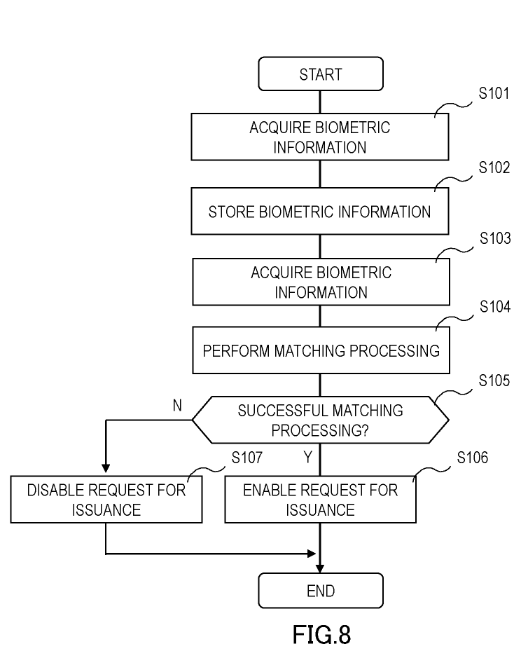

FIG. 8 is a flowchart illustrating an example of an operation of the identity verification unit 202. The operation of the identity verification unit 202 will be described with reference to FIG. 8.

The identity verification unit 202 acquires information related to the holder of an identity document possessed by the user at the time of opening the digital wallet or at the time of a request for issuance of digital contents (the medical checkup certificate VCs). For example, the identity verification unit 202 acquires biometric information of the holder of the identity document from an IC (Integrated Circuit) chip mounted in a My Number Card or a passport (Step S101).

For example, in a case where a My Number Card is used as an identity document, the identity verification unit 202 acquires a PIN for an electronic certificate for user proof by using a GUI (Graphical User Interface) and the like. Alternatively, in a case where a passport is used as an identity document, the identity verification unit 202 acquires information written in an MRZ (Machine Readable Zone) on the passport surface by using OCR (Optical Character Recognition) technology.

The identity verification unit 202 reads information from an IC chip by using the acquired PIN (a four-digit number) or the information written in the MRZ as a password. The identity verification unit 202 stores, in the storage unit 205, biometric information (face information, face image) related to the holder of the identity document read from the identity document (a My Number Card, a passport, and the like) (Step S102).

In addition, the identity verification unit 202 acquires biometric information of the user (a user of the terminal 30) (Step S103). For example, the identity verification unit 202 prompts the user to photograph his or her face by using a GUI and the like. For example, the identity verification unit 202 acquires a face image by so-called self-photographing.

In a case where the identity verification unit 202 acquires biometric information from an identity document and acquires biometric information of a user who operates the own apparatus, the identity verification unit 202 performs matching processing using the biometric information obtained from the identity document and the biometric information of the user (Step S104). The identity verification unit 202 determines whether the two biometric information items substantially match.

Specifically, the identity verification unit 202 generates feature values from each of the two biometric information items (for example, face images).

It should be noted that since an existing technology can be used to generate the feature values, a detailed description thereof will be omitted. For example, the identity verification unit 202 extracts eyes, a nose, a mouth, and the like as feature points from a face image. Thereafter, the identity verification unit 202 calculates, as feature values, the positions of the respective feature points and distances between the respective feature points (generates a feature vector composed of a plurality of feature values).

Next, the identity verification unit 202 performs matching processing (authentication processing) using the two generated feature values. Specifically, the identity verification unit 202 calculates a similarity between corresponding face images using the two feature values. The identity verification unit 202 determines whether the two images are face images of the same person based on a result of a threshold process applied to the calculated similarity. It should be noted that the chi-squared distance, the Euclidean distance, or the like can be used for the individual similarity. A longer distance represents a lower similarity, and a shorter distance represents a higher similarity.

For example, if the similarity is over a predetermined value (if the distance is shorter than a predetermined value), the identity verification unit 202 determines that the matching processing has succeeded. In a case where the similarity is equal to or less than the predetermined value, the identity verification unit 202 determines that the matching processing has failed.

In a case where the matching processing succeeds (Step S105, Yes branch), the identity verification unit 202 sets a state of the terminal 30 to enable a request for issuance of a certificate (the medical checkup certificate VCs) (Step S106).

In a case where the matching processing fails (Step S105, No branch), the identity verification unit 202 sets a state of the terminal 30 to disable a request for issuance of a certificate (the medical checkup certificate VCs) (Step S107).

In this manner, the identity verification unit 202 determines that identity verification has succeeded in a case where the authentication processing using biometric information obtained from an identity document and biometric information of a user of the digital wallet has succeeded. In a case where identity verification succeeds, the user becomes able to request issuance of a certificate from a certificate issuer (for example, a hospital).

The acquisition control unit 203 is means for performing control related to acquisition of credential certificates including the medical checkup certificate VCs.

For example, the acquisition control unit 203 requests issuance of the medical checkup certificate VCs to the hospital server 10. The acquisition control unit 203 stores the medical checkup certificate VCs acquired from the hospital server 10 in the digital wallet.

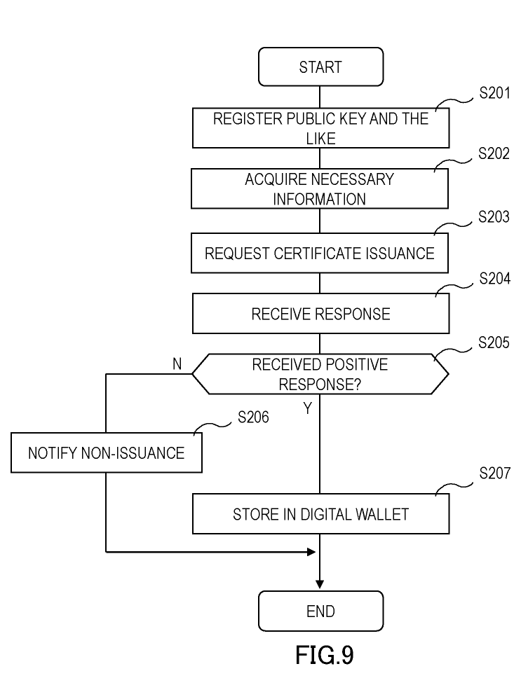

FIG. 9 is a flowchart illustrating an example of an operation of the acquisition control unit 203. With reference to FIG. 9, an operation of the acquisition control unit 203 according to the example embodiment of the present disclosure will be described.

In a case where a user launches the digital wallet application and performs a predetermined operation (for example, pressing a medical checkup certificate issuance button), the acquisition control unit 203 performs control related to acquisition of the medical checkup certificate VCs. At that time, in a case where identity verification of the user has succeeded, the acquisition control unit 203 performs control related to acquisition of the medical checkup certificate VCs.

First, the acquisition control unit 203 generates a pair of a public key and a private key and a user ID (a user DID), which is a decentralized identifier. The acquisition control unit 203 registers the generated user ID and the public key in a blockchain (registration of the public key and the like; Step S201).

Subsequently, the acquisition control unit 203 acquires information necessary for a request for issuance of the medical checkup certificate VCs by using a GUI and the like (acquisition of necessary information; Step S202).

For example, the acquisition control unit 203 acquires information related to a hospital having authority to issue the medical checkup certificate VCs (for example, a hospital name). Alternatively, the acquisition control unit 203 acquires information related to identification of a user by the hospital server 10 (for example, a patient ID number).

The acquisition control unit 203 notifies a certificate issuer of the acquired necessary information and the user ID. Specifically, the acquisition control unit 203 notifies the hospital server 10 of information related to identifying a certificate holder of the medical checkup certificate VCs (for example, a patient ID number) and the user ID. The acquisition control unit 203 transmits a "certificate issuance request," including information related to identification of a certificate holder and the user ID and the like, to the hospital server 10 (Step S203).

At that time, the acquisition control unit 203 may identify an address of the hospital server 10 to which the certificate issuance request is to be transmitted by referring to table information and the like that stores an association between a hospital name and an address of the hospital server 10.

It should be noted that the acquisition control unit 203 may add a signature to information included in a digital business card issuance request by using a private key corresponding to the public key registered in the blockchain.

The acquisition control unit 203 receives a response (a positive response, a negative response) to the certificate issuance request from the hospital server 10 (Step S204).

In a case where a negative response indicating that issuance of the medical checkup certificate VCs has failed is received (Step S205, No branch), the acquisition control unit 203 notifies the user of the fact that the medical checkup certificate VCs were not issued (notification of non-issuance; Step S206).

In a case where a positive response indicating that issuance of the medical checkup certificate VCs has succeeded is received (Step S205, Yes branch), the acquisition control unit 203 stores the medical checkup certificate VCs included in the positive response in the digital wallet (Step S207). At that time, the acquisition control unit 203 may notify the user of the fact that the medical checkup certificate VCs have been issued.

The acquisition control unit 203 may verify a signature attached to the medical checkup certificate VCs acquired from the hospital server 10. In this case, the acquisition control unit 203 acquires, from a blockchain, a public key corresponding to an issuer ID written in the medical checkup certificate VCs. The acquisition control unit 203 verifies the signature attached to the medical checkup certificate VCs by using the acquired public key, and, in a case where the verification succeeds, may store the medical checkup certificate VCs in the digital wallet.

In this manner, the acquisition control unit 203 operates as first acquisition means for acquiring the medical checkup certificate VCs, which are certificates certifying results of a medical checkup undergone by a user and whose contents can be verified online. In addition, the acquisition control unit 203 stores the acquired medical checkup certificate VCs in the digital wallet. At that time, in a case where verification of a signature of the acquired medical checkup certificate VCs succeeds, the acquisition control unit 203 may store the acquired medical checkup certificate VCs in the digital wallet.

The usage control unit 204 is means for performing control related to usage of digital contents (credential certificates) stored in the digital wallet.

In a case where a certificate provision request is received from the insurance server 20, the usage control unit 204 selects, from among a plurality of credential certificates stored in the digital wallet, a credential certificate (the medical checkup certificate VCs) designated by a life insurance company. Thereafter, the usage control unit 204 signs the selected medical checkup certificate VCs by using a private key corresponding to the user's DID (user ID).

It should be noted that the usage control unit 204 signs the medical checkup certificate VCs by using a private key generated at the time of issuance of the medical checkup certificate VCs requested to be provided by the life insurance company (a private key corresponding to the user ID).

In a case where the medical checkup certificate VCs designated by the life insurance company can be provided, the usage control unit 204 transmits a positive response including the medical checkup certificate VCs with a signature to the insurance server 20. In a case where the medical checkup certificate VCs designated by the life insurance company cannot be provided, the usage control unit 204 transmits a negative response indicating unavailability of the medical checkup certificate VCs to the insurance server 20.

The storage unit 205 is means for storing information necessary for operations of the terminal 30. For example, the storage unit 205 stores the medical checkup certificate VCs and the like by means of the digital wallet.

### Hospital Server

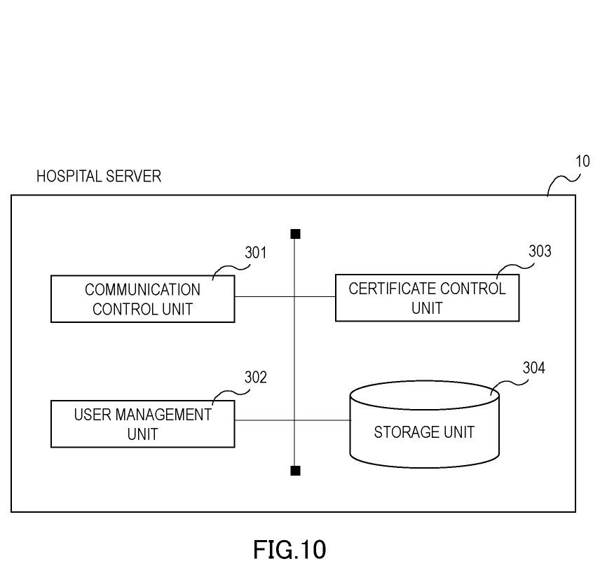

FIG. 10 is a diagram illustrating an example of a processing configuration (processing module) of the hospital server 10 according to an example embodiment of the present disclosure. Referring to FIG. 10, the hospital server 10 includes a communication control unit 301, a user management unit 302, a certificate control unit 303, and a storage unit 304.

The communication control unit 301 is means for controlling communication with other apparatuses. For example, the communication control unit 301 receives data (packets) from the terminal 30. In addition, the communication control unit 301 transmits data to the terminal 30. The communication control unit 301 gives data received from other apparatuses to other processing modules. The communication control unit 301 transmits data acquired from other processing modules to other apparatuses. In this way, other processing modules transmit and receive data to and from other apparatuses via the communication control unit 301. The communication control unit 301 includes a function as a receiving unit that receives data from other apparatuses and a function as a transmitting unit that transmits data to other apparatuses.

The user management unit 302 is means for performing management and the like of users of the hospital (patients, medical checkup recipients).

The user management unit 302 acquires user information (a name, a gender, a date of birth, an address, a patient ID number, a telephone number, an email address, and the like) from hospital staff and the like. In addition, the user management unit 302 acquires medical checkup results of the user.

The user management unit 302 stores the acquired information in a predetermined database.

The certificate control unit 303 is means for executing control related to the medical checkup certificate VCs. For example, the certificate control unit 303 issues the medical checkup certificate VCs to a user. The certificate control unit 303 processes a "certificate issuance request" received from the terminal 30.

In a case where the certificate issuance request is received, the certificate control unit 303 searches a database by using information

related to identifying the certificate holder included in the certificate issuance request (for example, a patient ID number) as a key.

In a case where the search fails (in a case where the corresponding user is not registered in the database), the certificate control unit 303 sends a negative response to the terminal 30 indicating a failure to issue the health check certificate VCs. Alternatively, in a case where medical checkup results are not stored in an entry identified by the search, the certificate control unit 303 transmits a negative response indicating failure in issuance of the medical checkup certificate VCs to the terminal 30.

In a case where the above-described search succeeds and medical checkup results are stored in the entry identified by the search, the certificate control unit 303 generates the medical checkup certificate VCs by using information stored in the database. The certificate control unit 303 generates the medical checkup certificate VCs including an issuer ID and a user ID (a DID of the certificate holder of the certificate; the user ID included in the certificate issuance request).

Specifically, the certificate control unit 303 generates, as the medical checkup certificate VCs, credential certificates including metadata such as a type of credential certificate, an issuing organization name, an issuance date and time, and the like, credential information itself, and information such as the issuer's public key information and an electronic signature. It should be noted that the credential information itself includes a hospital name, a name of the certificate holder of the medical checkup certificate VCs, medical checkup results, and the like. In addition, the electronic signature attached to the medical checkup certificate VCs is made by using a private key corresponding to an issuer ID generated in advance.

The certificate control unit 303 transmits the generated medical checkup certificate VCs to the terminal 30. Specifically, the certificate control unit 303 transmits a positive response including the medical checkup certificate VCs to the terminal 30. In addition, the certificate control unit 303 registers an issuer ID and a public key generated in advance, and the like, in a blockchain.

The storage unit 304 is means for storing information necessary for operations of the hospital server 10.

### Insurance Server

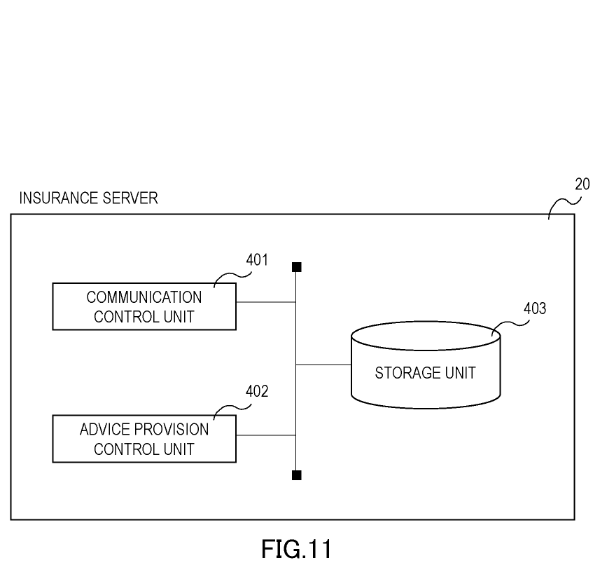

FIG. 11 is a diagram illustrating an example of a processing configuration (processing modules) of the insurance server 20 according to an example embodiment of the present disclosure. Referring to FIG. 11, the insurance server 20 includes a communication control unit 401, an advice provision control unit 402, and a storage unit 403.

The communication control unit 401 is means for controlling communication with other apparatuses. For example, the communication control unit 401 receives data (packets) from the terminal 30. In addition, the communication control unit 401 transmits data to the terminal 30. The communication control unit 401 gives data received from other apparatuses to other processing modules. The communication control unit 401 transmits data acquired from other processing modules to other apparatuses. In this way, other processing modules transmit and receive data to and from other apparatuses via the communication control unit 401. The communication control unit 401 includes a function as a receiving unit for receiving data from other apparatuses, and a function as a transmitting unit for transmitting data to other apparatuses.

The advice provision control unit 402 is means for executing control related to advice provided to a user.

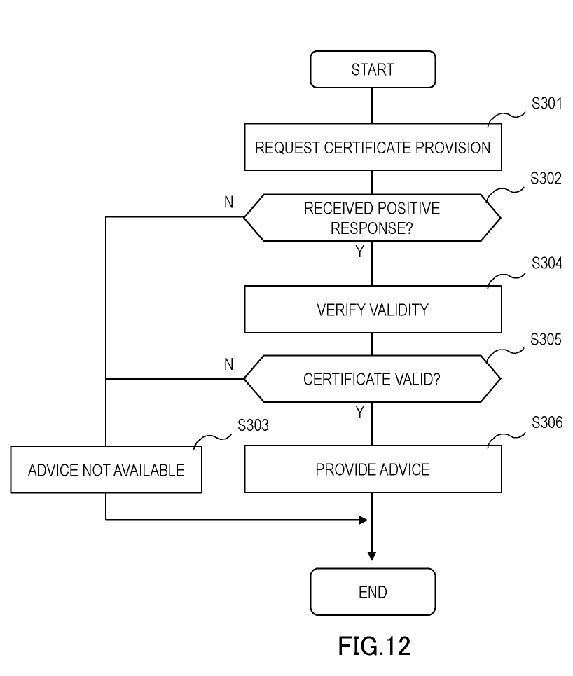

The operation of the advice provision control unit 402 will be described with reference to FIG. 12.

In a case where a user desires provision of advice, the advice provision control unit 402 acquires information necessary for generating the advice (the medical checkup certificate VCs).

Specifically, the advice provision control unit 402 transmits a "certificate provision request" to the terminal 30 (Step S301). The advice provision control unit 402 transmits to the terminal 30 the certificate provision request in which the medical checkup certificate VCs are set as information necessary for the advice.

In a case where a negative response (unavailability of the medical checkup certificate VCs) is received from the terminal 30 (Step S302, No branch), the advice provision control unit 402 notifies the user and the like that the advice cannot be provided due to failure to acquire necessary information (unavailability of advice provision; Step S303).

In a case where a positive response (a response including the medical checkup certificate VCs) is received from the terminal 30 (Step S302, Yes branch), the advice provision control unit 402 verifies validity of the medical checkup certificate VCs included in the positive response (verification of validity; Step S304).

The advice provision control unit 402 verifies at least one of three items related to validity of the medical checkup certificate VCs.

A first item is verification of an electronic signature attached to the medical checkup certificate VCs.

In this case, the advice provision control unit 402 acquires an issuer ID and a user ID written in the medical checkup certificate VCs. The advice provision control unit 402 acquires, from a blockchain, a public key corresponding to the acquired issuer ID. Similarly, the advice provision control unit 402 acquires, from the blockchain, a public key corresponding to the acquired user ID.

The advice provision control unit 402 verifies, respectively, a signature of a holder (a user who desires provision of advice) attached to the medical checkup certificate VCs and a signature of an issuer. Through verification of these signatures, the advice provision control unit 402 confirms that the medical checkup certificate VCs acquired from the user (the holder of the medical checkup certificate VCs) have not been tampered with and are certificates issued by a trusted issuer.

In a case where verification of both the holder's signature and the issuer's signature succeeds, the advice provision control unit 402 determines that the medical checkup certificate VCs are valid.

A second item is verification that the medical checkup certificate VCs have not been invalidated.

In this case, the advice provision control unit 402 accesses a blockchain and confirms that the medical checkup certificate VCs received are not listed in a revocation list of the certificate issuer. The advice provision control unit 402 confirms that the medical checkup certificate VCs acquired from the user have not been invalidated by the issuer before expiration of their validity period.

In a case where the medical checkup certificate VCs are not listed in the revocation list, the advice provision control unit 402 determines that the acquired medical checkup certificate VCs are valid.

A third item is verification that a validity period (expiration date) of the medical checkup certificate VCs has not elapsed.

The advice provision control unit 402 confirms a validity period set in the medical checkup certificate VCs. In a case where the validity period set in the medical checkup certificate VCs has not elapsed, the advice provision control unit 402 determines that the acquired medical checkup certificate VCs are valid.

The advice provision control unit 402 determines that the medical checkup certificate VCs acquired from the user are valid in a case where, in accordance with its own policy and the like, the medical checkup certificate VCs are determined to be valid in at least one predetermined item among the first to third items.

For example, in a case where the medical checkup certificate VCs are determined to be valid in all three items, the advice provision control unit 402 determines that the acquired medical checkup certificate VCs are valid (accepts the medical checkup certificate VCs). Alternatively, in a case where the medical checkup certificate VCs are determined to be valid in two items, namely the first item and the third item, the advice provision control unit 402 may determine that the acquired medical checkup certificate VCs are valid.

In a case where the medical checkup certificate VCs are not valid (Step S305, No branch), the advice provision control unit 402 notifies the user and the like that advice cannot be provided because the medical checkup certificate VCs are invalid (unavailability of advice provision; Step S303).

In a case where the medical checkup certificate VCs are valid (Step S305, Yes branch), the advice provision control unit 402 provides advice to the user (Step S306).

For example, the advice provision control unit 402 provides advice related to insurance products or advice related to the user's health based on the medical checkup results obtained from the medical checkup certificate VCs.

At that time, the advice provision control unit 402 may generate the above-described advice by using an AI (Artificial Intelligence) model obtained through machine learning. For example, the advice provision control unit 402 may generate advice by using generative AI (for example, a large language model). For example, the advice provision control unit 402 may generate a prompt instructing generation of health-related advice based on the medical checkup results, and may generate (acquire) advice by inputting the generated prompt into the large language model.

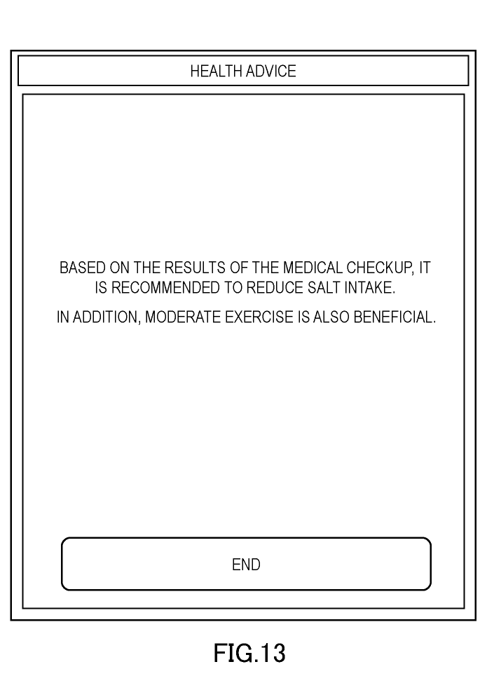

The advice provision control unit 402 outputs the generated advice. The advice provision control unit 402 displays the generated advice on the terminal 30. For example, the advice provision control unit 402 displays, on the terminal 30, a screen such as that illustrated in FIG. 13.

The storage unit 403 is means for storing information necessary for operations of the insurance server 20.

### Operation of System

Next, operations of the information processing system according to the first example embodiment will be described.

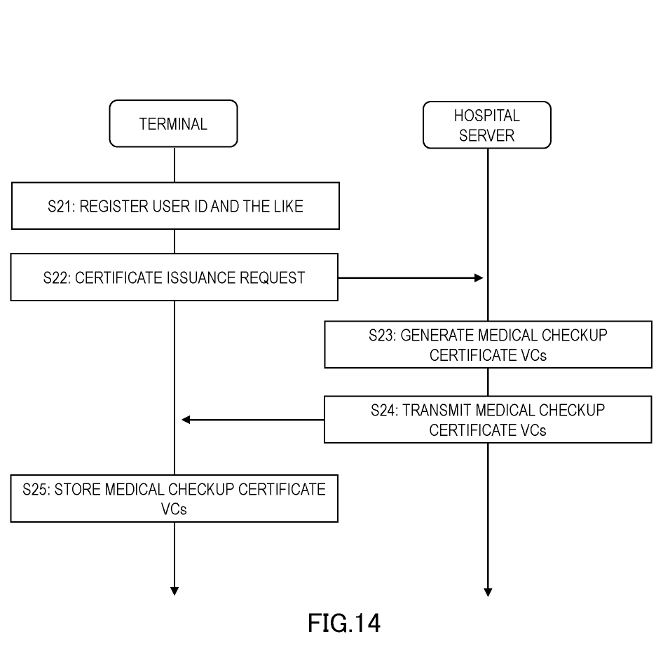

FIG. 14 is a sequence diagram illustrating an example of operations of the information processing system according to an example embodiment of the present disclosure. With reference to FIG. 14, operations related to issuance of the medical checkup certificate VCs in the information processing system according to the first example embodiment will be described.

The terminal 30 generates a public key, a private key, and a user ID, and registers the user ID and the public key in a blockchain (Step S21).

The terminal 30 transmits a certificate issuance request including the user ID to the hospital server 10 (Step S22).

The hospital server 10 generates credential information of the user (the certificate holder of the medical checkup certificate VCs) and generates medical checkup certificate VCs including the generated credential information (step S23). The hospital server 10 generates medical checkup certificate VCs including a user ID and an issuer ID and attached with an electronic signature.

The hospital server 10 transmits the generated medical checkup certificate VCs to the terminal 30 (step S24).

The terminal 30 stores the medical checkup certificate VCs in the digital wallet (step S25).

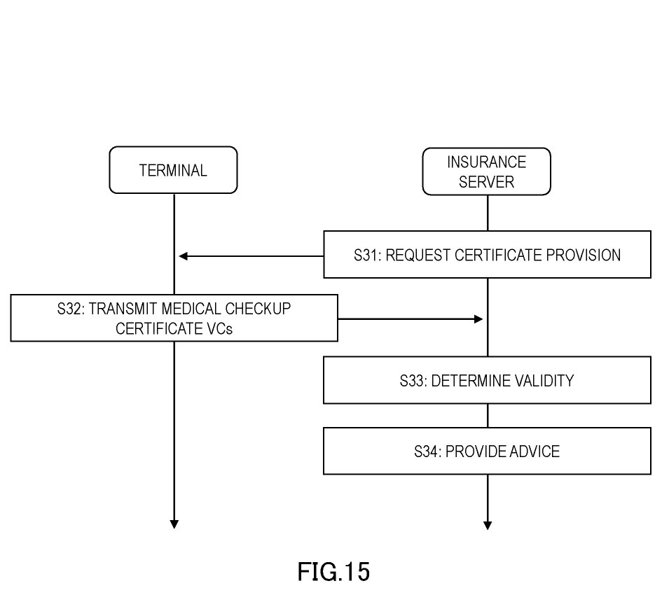

FIG. 15 is a sequence diagram illustrating an example of operation of the information processing system according to the example embodiment of the present disclosure. With reference to FIG. 15, operations related to use of the medical checkup certificate VCs in the information processing system according to the first example embodiment will be described.

The insurance server 20 of the life insurance company transmits to the terminal 30 a certificate provision request specifying information required for provision of advice (step S31).

The terminal 30 reads out from the digital wallet the medical checkup certificate VCs specified by the life insurance company and transmits the signed medical checkup certificate VCs to the insurance server 20 (step S32).

The insurance server 20 determines validity of the acquired medical checkup certificate VCs (step S33).

In a case where the medical checkup certificate VCs are valid, the insurance server 20 provides advice to the user (step S34).

As described above, the information processing system according to the first example embodiment includes an issuance means, an acquisition means, a verification means, and an output means. For example, a certificate control unit 303 of the hospital server 10 corresponds to the issuance means. In addition, an advice provision control unit 402 of the insurance server 20 corresponds to the acquisition means, the verification means, and the output means.

The issuance means issues to the user, in the form of VCs (Verifiable Credentials), a certificate that certifies predetermined matters (for example, a medical checkup certificate certifying medical checkup results). The acquisition means acquires a certificate (for example, a medical checkup certificate VCs) issued to the user in a case where the user requests the provision of advice from a service provider (for example, a life insurance company). The verification means performs verification on the certificate acquired by the acquisition means. The output means, in a case where verification of the certificate succeeds, generates advice (for example, advice regarding insurance products or health advice) based on predetermined matters certified by the certificate, and outputs the generated advice.

Alternatively, the information processing system may include an authentication means (for example, an identity verification unit 202 of the terminal 30). The authentication means authenticates the user who receives the provision of advice by using biometric information of the user who receives the provision of the advice.

Furthermore, the above certificate (the medical checkup certificate VCs) is stored in the terminal 30 possessed by the user, and the acquisition means may acquire the certificate by requesting the terminal 30 possessed by the user to provide the certificate. In addition, the issuance means may issue the certificate to the user in response to a request from the user.

In this manner, in the information processing system according to the first example embodiment, the service provider may be a life insurance company. In that case, the certificate certifies the results of a medical checkup received by the user, and the output means may output advice regarding insurance products or advice regarding the user's health based on the medical checkup results.

Next, variations according to the first example embodiment will be described.

### Variation 1

In the above example embodiment, the operation of the information processing system has been described by taking as an

example a case where a life insurance company provides advice to the user. However, an organization or the like that substitutes for the life insurance company may provide advice to the user. For example, a health insurance association or the like may provide advice to the user.

That is, the service provider in the present disclosure may be a health insurance association. In addition, the health insurance association may provide advice by using a medical checkup certificate VCs that certifies the results of a medical checkup received by the user. More specifically, a server apparatus of the health insurance association that corresponds to the insurance server 20 may output advice regarding insurance products or advice regarding the user's health based on the medical checkup results.

### Variation 2

The information processing system of the present disclosure may provide the user with advice other than advice related to insurance products and the like. For example, the information processing system of the present disclosure may be used in fields such as fitness, medical counseling, health product promotion, wearable device linkage, event recommendations, education and human resource development, electronic commerce (EC) and retail, and financial services.

For example, in a case where the information processing system is applied to the fitness field, a fitness service provider may use certificates (certification information) related to the user's health information or exercise history and provide advice related to personalized training programs or meal plans.

That is, the service provider is a fitness service provider. A server apparatus of the fitness service provider corresponding to the insurance server 20 acquires from the terminal 30 a certificate that certifies the user's health condition or exercise history. The server apparatus may output advice regarding the user's training program or meal plan based on the user's health condition or exercise history.

It should be noted that a hospital can issue a credential certificate that certifies a health condition. In addition, a fitness service provider can issue a credential certificate that certifies an exercise history.

The information processing system of the present disclosure can provide effective fitness services tailored to the user's latest health condition and the like.

### Variation 3

In providing a medical checkup certificate VCs to a service provider, a verifiable presentation (VP) may be provided to the counterpart instead of the medical checkup certificate VCs, which is a credential certificate. That is, part of the medical checkup certificate VCs issued by the hospital may be provided from the user (the holder) to the service provider (for example, a life insurance company) as a verifiable presentation.

It should be noted that, in the following description, a medical checkup certificate provided as a VP (verifiable presentation) is referred to as a "medical checkup certificate VP."

For example, the user determines the items included in the medical checkup certificate VP according to the type of advice to be received. For example, in a case where the user desires to receive advice related to the selection of insurance products, the user provides to the life insurance company a medical checkup certificate VP that includes all information contained in the medical checkup certificate VP. In contrast, in a case where the user desires to receive health advice, the user provides to the life insurance company a medical checkup certificate VP that includes the medical checkup results and does not include the name, email address, and the like. That is, the terminal 30 may implement selective minimal disclosure using a VP (verifiable presentation).

It should be noted that, in order to implement the above selective minimal disclosure, the issuer of the medical checkup certificate VCs (the hospital) separates the data to be subject to selective minimal disclosure from the data set and calculates a hash value of the separated data. The hospital server 10 embeds the hash value of the separated data into the above data set. The hospital server 10 signs the entire data set including the hash value of the separated data and generates the medical checkup certificate VCs.

In providing the medical checkup certificate to the life insurance company, the usage control unit 204 of the terminal 30 displays a GUI that allows the selection of items to be provided to the life insurance company from among multiple items described in the medical checkup certificate VCs. For example, the usage control unit 204 acquires, using the GUI, items within the medical checkup certificate VCs that the user permits to provide to the life insurance company.

In this manner, the usage control unit 204 of the terminal 30 generates a verifiable presentation (a medical checkup certificate VP) that includes items selected by the user from among multiple items included in the user's medical checkup certificate VCs. The usage control unit 204 may provide the generated verifiable presentation to an external party.

As described above, in the information processing system according to the first example embodiment, the service provider (for example, a life insurance company) can provide advice to the user based on matters guaranteed by the credential certificate issued to the user (for example, medical checkup results). That is, the service provider can provide advice to the user based on reliable and accurate information certified by the credential certificate. As a result, more appropriate advice is provided to the user.

In addition, existing health information management systems and the like have a problem in that the verification process for health information is complex and time-consuming. In particular, in a case where a verifier needs to inquire of an issuer about certification information, delays and inefficiencies in the process were conspicuous. However, in the information processing system of the present disclosure, the verification process for certification information is greatly simplified, and time reduction is achieved. That is, an inquiry about certification information from the verifier to the issuer becomes unnecessary, and the verifier becomes able to perform immediate verification of the verification information. In addition, the introduction of biometric authentication improves security and strengthens the protection of the user's personal information.

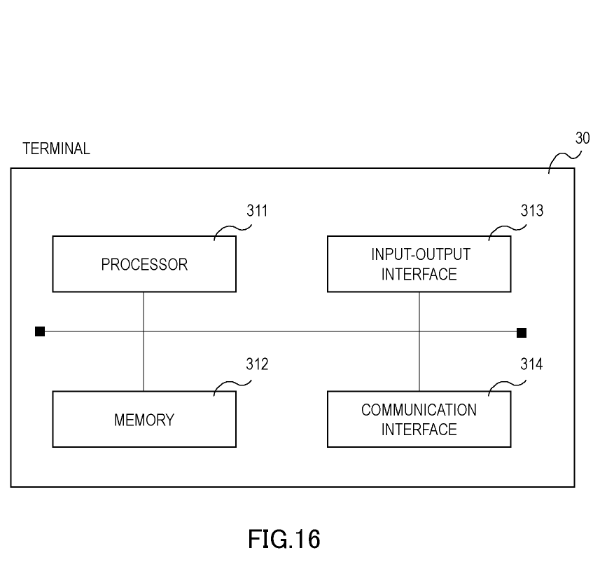

Next, a hardware configuration of an individual apparatus that constitutes the information processing system will be described. FIG. 16 is a diagram illustrating an example of a hardware configuration of the terminal 30.

The terminal 30 can be configured by an information processing apparatus (a so-called computer) and includes a configuration illustrated as an example in FIG. 16. For example, the terminal 30 includes a processor 311, a memory 312, an input-output interface 313, a communication interface 314, and the like. The above-described components such as the processor 311 are connected via an internal bus or the like and are configured to be capable of communicating with each other.

However, the hardware configuration of the terminal 30 is not limited to the configuration illustrated in FIG. 16. The terminal 30 may include hardware not illustrated or may be configured without the input-output interface 313 if desired. In addition, the number of components, such as the number of processors 311, included in the terminal 30 is not limited to the example illustrated in FIG. 16. For example, a plurality of processors 311 may be included in the terminal 30..

For example, the processor 311 is a programmable device such as a CPU (Central Processing Unit), an MPU (Micro Processing Unit), or a DSP (Digital Signal Processor). Alternatively, the processor 311 may be a device such as an FPGA (Field Programmable Gate Array) or an ASIC (Application Specific Integrated Circuit). The processor 311 executes various kinds of programs including an operating system (OS).

The memory 312 is a RAM (Random Access Memory), a ROM (Read-Only Memory), an HDD (Hard Disk Drive), an SSD (Solid State Drive), and the like. The memory 312 stores an OS program, an application program, and various kinds of data.

The input-output interface 313 is an interface for a display apparatus and an input apparatus not illustrated. The display apparatus is, for example, a liquid crystal display or the like. For example, the input apparatus is an apparatus that receives user operations, and examples of the input apparatus include a keyboard and a mouse.

The communication interface 314 is a circuit, a module, or the like for performing communication with other apparatuses. For example, the communication interface 314 includes a NIC (Network Interface Card) or the like.

The function of the terminal 30 is realized by various kinds of processing modules. The processing modules are realized, for example, by causing the processor 311 to execute a program stored in the memory 312. In addition, this program can be recorded in a computer-readable storage medium. The storage medium may be a non-transient (non-transitory) storage medium, such as a semiconductor memory, a hard disk, a magnetic recording medium, or an optical recording medium. That is, the present disclosure can be embodied as a computer program product. In addition, the above program may be updated by downloading a program via a network or by using a storage medium in which a program is stored. In addition, the above processing modules may be realized by semiconductor chips.

It should be noted that the hospital server 10 and the insurance server 20 can also be configured by an information processing apparatus similar to the terminal 30, and their basic hardware configurations are not different from that of the terminal 30, so their descriptions will be omitted.

The terminal 30, which is an information processing apparatus, includes a computer and can realize the functions of the terminal 30 by causing the computer to execute a program. In addition, the terminal 30 executes a terminal control method by using this program. Similarly, the hospital server 10 and the insurance server 20 include a computer, and the functions of the hospital server 10 and the like can be implemented by causing the computer to execute a program. In addition, the hospital server 10 and the like execute a control method of the hospital server 10 by using the program.

### Variations

It should be noted that the configurations, operations, and the like of the information processing system according to the above example embodiments are examples and do not limit the present system configuration, and the like.

In the above example embodiment, a case was described in which, in issuing a request for the issuance of the medical checkup certificate VCs, the terminal 30 transmits a clinical card number to the hospital server 10 as information related to identifying the user. However, the terminal 30 may transmit biometric information (a face image) of the user to the hospital server 10 as information related to identifying the user. The hospital server 10 may identify the user requesting issuance of the medical checkup certificate VCs by performing matching processing (authentication processing) using the biometric information.

In the above example embodiment, a case has been described in which the hospital server 10 of the certificate issuer (hospital) issues a credential certificate that does not require a certification authority for verifying the certificate. However, the hospital server 10 may issue a certificate that requires a certification authority (a certificate based on a public key infrastructure).

In the above example embodiment, a case has been described in which identity verification is performed using biometric information obtained from an identification document, confirming that the certificate holder and the digital wallet user match. However, the identity verification may alternatively be performed using a digital certificate stored in the identification document. Specifically, the terminal 30 acquires a digital certificate (a signature-use digital certificate, a user-verification digital certificate) from the My Number Card possessed by the user. The terminal 30 transmits the acquired digital certificate to a certification authority (J-LIS: Japan Agency for Local Authority Information Systems) and requests the certification authority to verify the validity of the digital certificate. The terminal 30 determines that identity verification has succeeded in a case where the digital certificate is valid.

Some of the functions of each apparatus described above may be implemented in another device, apparatus, and the like. More specifically, the "identity verification unit (identity verification means)," the "acquisition control unit (acquisition control means)," the "advice providing control unit (advice providing control means)," and the like described above may be implemented in any of the apparatuses included in the system.

While the data exchange between each apparatus (for example, the hospital server 10 and the terminal 30) is not limited to any particular mode, data exchanged between these apparatuses may be encrypted. It is desirable that personal information of a user, and so on are transmitted and received between these apparatuses and encrypted data is transmitted and received in order to properly protect this information.

In the flowcharts and sequence diagrams used in the above description, a plurality of steps (processes) are sequentially described. However, the order of the execution of the steps performed in the individual example embodiment is not limited to the described order. In the individual example embodiment, the order of the illustrated steps may be changed to the extent that a problem is not caused on the content of the individual example embodiment. For example, individual processes may be executed in parallel.

The above example embodiments have been described in detail to facilitate the understanding of the present application disclosed and not to mean that all the configurations described above are needed. In addition, if a plurality of example embodiments have been described, each of the example embodiments may be used individually or a plurality of example embodiments may be used in combination. For example, part of a configuration according to one example embodiment may be replaced by a configuration according to another example embodiment. For example, a configuration according to one example embodiment may be added to a configuration according to another example embodiment. In addition, addition, deletion, or replacement is possible between part of a configuration according to one example embodiment and another configuration.

The industrial applicability of the present disclosure has been made apparent by the above description. That is, the present disclosure is suitably applicable, for example, to an information processing system that utilizes credential certificates stored in a digital wallet and the like.

The information processing system according to the present disclosure provides specific technical improvements beyond an abstract idea. In particular, the information processing system according to the present disclosure exhibits clear improvements over conventional technologies in the secure management of certification information (certificate) and in efficient verification processes. A specific example includes the sharing and verification processes of patient data between medical institutions and insurance companies. By using the information processing system according to the present disclosure, the patient's (user's) medical records are securely issued in the form of credential certificates, and the life insurance company becomes able to verify the medical records immediately. As a result, the processing flow such as advice related to insurance products is significantly accelerated, and administrative efficiency is improved. At the same time, strict identity verification using biometric authentication reduces the risk of medical fraud. In this manner, the present disclosure is not merely an abstract idea but provides a practical application that solves real-world problems using concrete technical means.

A part or the entirety of the example embodiments described above may be described as in the following supplementary notes, but is not limited to the followings.

### Supplementary Note 1

A system including:

an issuance means that issues, to a user, a certificate that certifies predetermined matters in a VCs (Verifiable Credentials) format;

an acquisition means that acquires the certificate issued to the user in a case where the user requests provision of advice from a service provider;

a verification means that verifies the acquired certificate; and

an output means that generates, in a case where verification of the certificate succeeds, the advice based on the predetermined matters that the certificate certifies, and outputs the generated advice.

### Supplementary Note 2

The system according to supplementary note 1, further including an authentication means that authenticates the user who receives the provision of the advice by using biometric information of the user who receives the provision of the advice.

### Supplementary Note 3

The system according to supplementary note 2, wherein the certificate is stored in a terminal possessed by the user, and

wherein the acquisition means acquires the certificate by requesting the terminal possessed by the user to provide the certificate.

### Supplementary Note 4

The system according to supplementary note 3, wherein the issuance means issues the certificate to the user in response to the request from the user.

### Supplementary Note 5

The system according to any one of supplementary notes 1 to 4, wherein the service provider is a life insurance company, and

wherein the certificate certifies results of a medical checkup received by the user, and

wherein the output means outputs the advice regarding insurance products or the advice regarding user's health based on the results of the medical checkup.

### Supplementary Note 6

The system according to any one of supplementary notes 1 to 4, wherein the service provider is a health insurance association, and

wherein the certificate certifies results of a medical checkup received by the user, and

wherein the output means outputs the advice regarding insurance products or the advice regarding user's health based on the results of medical checkup.

### Supplementary Note 7

The system according to any one of supplementary notes 1 to 4, wherein the service provider is a fitness service provider, and

wherein the certificate certifies user's health condition or exercise history, and

wherein the output means outputs the advice regarding user's training program or meal plan based on the user's health condition or exercise history.

### Supplementary Note 8

A server apparatus including:

an acquisition means that acquires a certificate, the certificate certifying predetermined matters and being issued in a VCs (Verifiable Credentials) format, in a case where a user requests provision of advice from a service provider;

a verification means that verifies the acquired certificate; and

an output means that generates, in a case where verification of the certificate succeeds, the advice based on the predetermined matters that the certificate certifies, and outputs the generated advice.

### Supplementary Note 9

A control method of a server apparatus, the control method including:

acquiring a certificate, the certificate certifying predetermined matters and being issued in a VCs (Verifiable Credentials) format, in a case where a user requests provision of advice from a service provider;

verifying the acquired certificate;

generating, in a case where verification of the certificate succeeds, the advice based on the predetermined matters that the certificate certifies; and

outputting the generated advice.

### Supplementary Note 10

A program causing a computer mounted on a server apparatus to perform processing for:

acquiring a certificate, the certificate certifying predetermined matters and being issued in a VCs (Verifiable Credentials) format, in a case where a user requests provision of advice from a service provider;

verifying the acquired certificate;

generating, in a case where verification of the certificate succeeds, the advice based on the predetermined matters that the certificate certifies; and

outputting the generated advice.

In addition, part or all of the configurations described in supplementary notes 2 to 7, which depend on supplementary note 1 as described above, may also depend on supplementary notes 8 to 10 in a dependency relationship similar to that of supplementary notes 2 to 7. Furthermore, not limited to supplementary note 1 and supplementary notes 9 and 17, within a range not departing from the above-described example embodiments, a part or all of the configurations described as supplementary notes can likewise be made dependent on various hardware, software, various recording means for recording the software, or systems.

The entire disclosure of the above patent literature is incorporated herein by reference thereto. While the example embodiments of the present disclosure have thus been described, the present disclosure is not limited to these example embodiments. It is to be understood to those skilled in the art that these example embodiments are only examples and that various variations are possible without departing from the scope and spirit of the present disclosure. That is, the present disclosure of course includes various variations and modifications that could be made by those skilled in the art in accordance with the overall disclosure including the claims and the technical concept.

The previous description of embodiments is provided to enable a person skilled in the art to make and use the present disclosure. Moreover, various modifications to these example embodiments will be readily apparent to those skilled in the art, and the generic principles and specific examples defined herein may be applied to other embodiments without the use of inventive faculty. Therefore, the present disclosure is not intended to be limited to the example embodiments described herein but is to be accorded the widest scope as defined by the limitations of the claims and equivalents. Further, it is noted that

the inventor's intent is to retain all equivalents of the claimed disclosure even if the claims are amended during prosecution.

What is claimed is:

1. A system comprising:
at least one memory storing a set of instructions; and
at least one processor configured to execute the set of instructions to:
issue, to a user, a certificate that certifies predetermined matters in a VCs (Verifiable Credentials) format;
acquire the certificate issued to the user in a case where the user requests provision of advice from a service provider;
verify the acquired certificate;
generate, in a case where verification of the certificate succeeds, the advice based on the predetermined matters that the certificate certifies; and
output the generated advice.

2. The system according to claim 1, wherein
the at least one processor is further configured to execute the set of instructions to authenticate the user who receives the provision of the advice by using biometric information of the user who receives the provision of the advice.

3. The system according to claim 2, wherein the certificate is stored in a terminal possessed by the user, and
wherein the at least one processor is further configured to execute the set of instructions to acquire the certificate by requesting the terminal possessed by the user to provide the certificate.

4. The system according to claim 3, wherein
the at least one processor is further configured to execute the set of instructions to issue the certificate to the user in response to the request from the user.

5. The system according to claim 1, wherein the service provider is a life insurance company, and
wherein the certificate certifies results of a medical checkup received by the user, and
wherein the at least one processor is further configured to execute the set of instructions to output the advice regarding insurance products or the advice regarding user's health based on the results of the medical checkup.

6. The system according to claim 2, wherein the service provider is a life insurance company, and
wherein the certificate certifies results of a medical checkup received by the user, and
wherein the at least one processor is further configured to execute the set of instructions to output the advice regarding insurance products or the advice regarding user's health based on the results of the medical checkup.

7. The system according to claim 3, wherein the service provider is a life insurance company, and
wherein the certificate certifies results of a medical checkup received by the user, and
wherein the at least one processor is further configured to execute the set of instructions to output the advice regarding insurance products or the advice regarding user's health based on the results of the medical checkup.

8. The system according to claim 4, wherein the service provider is a life insurance company, and
wherein the certificate certifies results of a medical checkup received by the user, and
wherein the at least one processor is further configured to execute the set of instructions to output the advice regarding insurance products or the advice regarding user's health based on the results of the medical checkup.

9. The system according to claim 1, wherein the service provider is a health insurance association, and
wherein the certificate certifies results of a medical checkup received by the user, and
wherein the at least one processor is further configured to execute the set of instructions to output the advice regarding insurance products or the advice regarding user's health based on the results of medical checkup.

10. The system according to claim 2, wherein the service provider is a health insurance association, and
wherein the certificate certifies results of a medical checkup received by the user, and
wherein the at least one processor is further configured to execute the set of instructions to output the advice regarding insurance products or the advice regarding user's health based on the results of medical checkup.

11. The system according to claim 3, wherein the service provider is a health insurance association, and
wherein the certificate certifies results of a medical checkup received by the user, and
wherein the at least one processor is further configured to execute the set of instructions to output the advice regarding insurance products or the advice regarding user's health based on the results of medical checkup.

12. The system according to claim 4, wherein the service provider is a health insurance association, and
wherein the certificate certifies results of a medical checkup received by the user, and
wherein the at least one processor is further configured to execute the set of instructions to output the advice regarding insurance products or the advice regarding user's health based on the results of medical checkup.

13. The system according to claim 1, wherein the service provider is a fitness service provider, and
wherein the certificate certifies user's health condition or exercise history, and
wherein the at least one processor is further configured to execute the set of instructions to output the advice regarding user's training program or meal plan based on the user's health condition or exercise history.

14. A server apparatus comprising:
at least one memory storing a set of instructions; and
at least one processor configured to execute the set of instructions to:
acquire a certificate, the certificate certifying predetermined matters and being issued in a VCs (Verifiable Credentials) format, in a case where a user requests provision of advice from a service provider;
verify the acquired certificate;
generate, in a case where verification of the certificate succeeds, the advice based on the predetermined matters that the certificate certifies; and
output the generated advice.

15. A control method of a server apparatus, the control method comprising:
acquiring a certificate, the certificate certifying predetermined matters and being issued in a VCs (Verifiable Credentials) format, in a case where a user requests provision of advice from a service provider;
verifying the acquired certificate;
generating, in a case where verification of the certificate succeeds, the advice based on the predetermined matters that the certificate certifies; and
outputting the generated advice.
